(19) United States
(12) Patent Application Publication
(10) Pub. No.: US 2008/0305527 A1
(43) Pub. Date: Dec. 11, 2008

(54) Single Chain Pan ECTO-NOX Variable Region (ScFv) Antibody, Coding Sequence and Methods

(75) Inventor: Chinpal Kim, West Lafayette, IN (US)

Correspondence Address: GREENLEE WINNER AND SULLIVAN P C, 4875 PEARL EAST CIRCLE, SUITE 200, BOULDER, CO, 80301, US

(73) Assignee: NOX TECHNOLOGIES, INC., WEST LAFAYETTE, IN (US)

(21) Appl. No.: 11/849,170

(22) Filed: Aug. 31, 2007

Related U.S. Application Data
(60) Provisional application No. 60/824,398, filed on Sep. 1, 2006.

(51) Int. Cl. C12P 21/04; C12N 15/11; C12N 15/00; C07K 1/00; C12N 5/06

(52) U.S. Cl. 435/69.6; 536/23.53; 435/320.1; 435/325; 530/402

(57) ABSTRACT

The present Specification describes compositions and methods for production of a recombinant single chain variable region (ScFv) antibody useful for the detection of members of the ECTO-NOX family of cell surface proteins on Western blots. This single chain antibody is especially useful in the detection, diagnosis and monitoring of neoplastic disorders. A linker sequence required to appropriately combine the light and heavy chain variable region fragments to form the functional recombinant single chain antibodies is also provided. The resultant ScFv is a pan ECTO-NOX antibody for detection on Western blots of most if not all tNOX isoforms.

### CROSS-REFERENCE TO RELATED APPLICATIONS

This application claims benefit of U.S. Provisional Application 60/824,398, filed Sep. 1, 2006, which application is incorporated herein to the extent there is no inconsistency with the present disclosure.

### STATEMENT REGARDING FEDERALLY SPONSORED RESEARCH OR DEVELOPMENT

Not Applicable

### BACKGROUND OF THE INVENTION

The invention relates generally to recombinant single chain antibodies with utility in combinations with other techniques for detection of serum markers characteristic of diseased cells, specifically cell surface NADH:protein disulfide reductase (ECTO-NOX) isoforms, to nucleic acid molecules encoding single chain antibodies specific for the cell surface marker characteristic of neoplastic and certain other diseased cells, to recombinant cells producing those neoplastic marker-specific monoclonal antibodies and single chain antibodies of similar or different specificity,

There is a unique, growth-related family of cell surface hydroquinone or NADH oxidases with protein disulfide-thiol interchange activity referred to as ECTO-NOX proteins (for cell surface NADH oxidases) (1,2). One member of the ECTO-NOX family, designated tNOX (for tumor associated) is specific to the surfaces of cancer cells and the sera of cancer patients (3, 4). The presence of the tNOX protein has been demonstrated for several human tumor tissues (mammary carcinoma, prostate cancer, neuroblastoma, colon carcinoma and melanoma) (5), and serum analysis suggest a much broader association with human cancer (6, 7).

NOX proteins are ectoproteins anchored in the outer leaflet of the plasma membrane (8). As is characteristic of other examples of ectoproteins (sialyl and galactosyl transferase, dipeptidylamino peptidase IV, etc.), the NOX proteins are shed. They appear in soluble form in conditioned media of cultured cells (5) and in patient sera (6, 7). The serum form of tNOX from cancer patients exhibits the same degree of drug responsiveness as does the membrane-associated form. Drug-responsive tNOX activities are seen in sera of a variety of human cancer patients, including patients with leukemia, lymphomas or solid tumors (prostate, breast, colon, lung, pancreas, ovarian, liver) (6, 7). An extreme stability and protease resistance of the tNOX protein (9) may help explain its ability to accumulate in sera of cancer patients to readily detectable levels. In contrast, no drug-responsive NOX activities have been found in the sera of healthy volunteers (6, 7) or in the sera of patients with disorders other than neoplasia.

While the basis for the cancer specificity of cell surface tNOX has not been determined, the concept is strongly supported by several lines of evidence. A drug responsive tNOX activity has been rigorously determined to be absent from plasma membranes of non-transformed human and animal cells and tissues (3). The tNOX proteins lack a transmembrane binding domain (10) and are released from the cell surface by brief treatment at low pH (9). A drug-responsive tNOX activity has not been detected in sera from healthy volunteers or patients with diseases other than cancer (6, 7). Several tNOX antisera have identified the immunoreactive band at 34 kDa (the processed molecular weight of one of the cell surface forms of tNOX) with Western blot analysis or immunoprecipitation when using transformed cells and tissues or sera from patients with cancers as antigen source (5, 10, 11). No immunoreactive 34 kDa band was observed with Western blot analysis or immunoprecipitation when using non-transformed cells or tissue or sera from healthy volunteers or patients with disorders other than cancers as antigen source (5, 10, 11). Those antisera include a monoclonal antibody (5), single-chain variable region fragment (ScFv) which reacts with a cell surface NADH oxidase from neoplastic cells, polyclonal antisera made in response to expressed tNOX (11) and polyclonal peptide antisera to the conserved adenine nucleotide binding region of tNOX (11).

tNOX cDNA has been cloned (GenBank Accession No. AF207881; 11; US Patent Publication 2003-0207340 A1). The derived molecular weight from the open reading frame was 70.1 kDa. Functional motifs include a quinone binding site, an adenine nucleotide binding site, and a CXXXXC cysteine pair as a potential protein disulfide-thiol interchange site based on site-directed mutagenesis (11). Based on available genomic information (12) the tNOX gene is located on chromosome X, and it is comprised of multiple exons (thirteen). It is known that there are a number of splice variant mRNAs and proteins expressed.

The hybridoma cell line which produces the tumor NADH oxidase-specific monoclonal antibody MAB 12.1 was deposited with the American Type Culture Collection, Manassas, Va., 20108 on Apr. 4, 2002, under the terms of the Budapest Treaty. This deposit is identified by Accession No. ATCC PTA-4206. The deposit will be maintained with restriction in the ATCC depository for a period of 30 years from the deposit date, or 5 years after the most recent request, or for the effective life of the patent, which ever is longer, and will be replaced if the deposit becomes non-viable during that period. This monoclonal antibody is described in U.S. Pat. No. 7,053,188, issued May 30, 2006, incorporated by reference herein.

The light and heavy chain variable regions of DNA upon which ScFv production is based are from the monoclonal antibody produced by the hybridoma on deposit with the American Type Culture Collection as Accession No. PTA-4206. See also U.S. Pat. No. 7,053,188.

Because cancer poses a significant threat to human health and because cancer results in significant economic costs, there is a long-felt need in the art for an effective, economical and technically simple system in which to assay for the presence of cancer, and for use in such an assay, an conveniently replenished source of the specific antibody can identify proteins characteristic of neoplastic conditions.

### BRIEF SUMMARY OF THE INVENTION

The present invention provides a recombinant single chain antibody specific for Ecto-NOX, coding sequences therefore and methods for recombinant production of this single chain antibody. This recombinant antibody is useful for the analysis of a biological sample for the presence of particular isoforms of the pan-cancer antigen known as tNOX (for tumor-specific NADH oxidase) and other members of the ECTO-NOX family of proteins in Western blots. The amino acid sequence of the single chain antibody specific for Ecto-NOX proteins, including tNOX, is presented in FIG. 4B, and a specifically exemplified coding sequence is given in FIG. 4A; it is understood that synonymous coding sequences are within the scope of the present invention, and functionally equivalent single chain antibodies with the same binding specificity are also within the scope of the present invention.

The single chain variable region antibody (ScFv) allows for identification of ECTO-NOX isoforms on Western blots was demonstrated to have utility in cancer detection, diagnosis and monitoring of disease progression. The detection of tNOX isoforms on Western blots of 2-D gels of patient sera, is described in US provisional patent application 60/824,333, filed September 1, 2006. “Neoplasia Specific tNOX Isoforms and Methods” which is incorporated by reference herein.

### BRIEF DESCRIPTION OF THE DRAWINGS

FIG. 1A and SEQ ID NO:1 provide the sequence encoding the Ecto-NOX specific heavy chain ScFv (V_{H}), and FIG. 1B and SEQ ID NO:2 provide the DNA sequence encoding the Ecto-NOX specific light chain ScFv (V_{L}).

FIG. 2 presents the strategy for the synthesis of the ScFv gene.

FIG. 3 is a diagram of the pET-11a expression vector (available from Stratagene, La Jolla, Calif.). The sequence of the multiple cloning site portion is also shown (SEQ ID NO:17), as is the N-terminal sequence of the T7 gene 10 leader peptide (SEQ ID NO:18).

FIGS. 4A and 4B provide the DNA coding sequence and the deduced amino acid sequences of ScFv(S), respectively. See also SEQ ID NO:3 and SEQ ID NO:4, respectively.

### DETAILED DESCRIPTION OF THE INVENTION

The present invention provides a recombinant single chain antibody which specifically binds all Ecto-NOX proteins, including the tumor specific form associated with tumor and other neoplastic cells (tNOX), when these proteins are resolved by gel electrophoresis and blotted to a solid support (such as a nylon support) (Western blot, immunoblot analysis).

The expression and function of the single chain antibody of the present invention for use in Western blots has been found to be dependent on the particular linker which joins the variable heavy and variable light portions. As specifically exemplified, the sequence of the linker is GGGGSGGGGSGGGGS (SEQ ID NO:5). The specifically exemplified single chain antibody also contains, at its C-terminus, a so-called S-tag, an amino acid sequence which allows for the use of a second antibody which specifically binds to this S-protein sequence, thus facilitating detection of the binding of the single chain antibody to an immunoblot.

As an alternative to the specifically exemplified S-tag, it is understood that other tag sequences which serve as binding sites for detectable second antibodies or other detectable molecule (with binding specificity for the single chain antibody of the present invention) or to facilitate purification over an affinity column or an immunoaffinity column can be substituted for the S tag. These tags, and expression vectors containing them include without limitation, the GST (glutathione S-transferase), flagellar antigen, Myc, Nus, among others. Other oligopeptide “tags” which can be fused to a protein of interest by molecular biological techniques include, without limitation, strep-tag (Sigma-Genosys, The Woodlands, Tex.) which directs binding to streptavidin or its derivative streptactin (Sigma-Genosys); a glutathione-S-transferase gene fusion system which directs binding to glutathione coupled to a solid support (Amersham Pharmacia Biotech, Uppsala, Sweden); a calmodulin-binding peptide fusion system which allows purification using a calmodulin resin (Stratagene, La Jolla, Calif.); a maltose binding protein fusion system allowing binding to an amylose resin (New England Biolabs, Beverly, Mass.); and an oligo-histidine fusion peptide system which allows purification using a Ni2+-NTA column (Qiagen, Valencia, Calif.).

The amino acids which occur in the various amino acid sequences referred to in the specification have their usual three- and one-letter abbreviations routinely used in the art: A, Ala, Alanine; C, Cys, Cysteine; D, Asp, Aspartic Acid; E, Glu, Glutamic Acid; F, Phe, Phenylalanine; G, Gly, Glycine; H, His, Histidine; I, Ile, Isoleucine; K, Lys, Lysine; L, Leu, Leucine; M, Met, Methionine; N, Asn, Asparagine; P, Pro, Proline; Q, Gln, Glutamine; R, Arg, Arginine; S, Ser, Serine; T, Thr, Threonine; V, Val, Valine; W, Try, Tryptophan; Y, Tyr, Tyrosine.

A protein is considered an isolated protein if it is a protein isolated from a host cell in which it is recombinantly produced. It can be purified or it can simply be free of other proteins and biological materials with which it is associated in nature.

An isolated nucleic acid is a nucleic acid the structure of which is not identical to that of any naturally occurring nucleic acid or to that of any fragment of a naturally occurring genomic nucleic acid spanning more than three separate genes. The term therefore covers, for example, (a) a DNA which has the sequence of part of a naturally occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transformed or transfected cells, and (iii) cell clones, e.g., as these occur in a DNA library such as a cDNA or genomic DNA library.

As used herein expression directed by a particular sequence is the transcription and translation of an associated downstream sequence.

In the present context, a promoter is a DNA region which includes sequences sufficient to cause transcription of an associated (downstream) sequence. The promoter may be regulated, i.e., not constitutively acting to cause transcription of the associated sequence. If inducible, there are sequences present which mediate regulation of expression so that the associated sequence is transcribed only when an inducer molecule is present in the medium in or on which the organism is cultivated.

In the present context, a transcription regulatory sequence includes a promoter sequence and can further include cis-active sequences for regulated expression of an associated sequence in response to environmental signals.

One DNA portion or sequence is downstream of second DNA portion or sequence when it is located 3′ of the second sequence. One DNA portion or sequence is upstream of a second DNA portion or sequence when it is located 5′ of that sequence.

One DNA molecule or sequence and another are heterologous to another if the two are not derived from the same ultimate natural source. The sequences may be natural sequences, or at least one sequence can be designed by man, as in the case of a multiple cloning site region. The two sequences can be derived from two different species or one sequence can be produced by chemical synthesis provided that the nucleotide sequence of the synthesized portion was not derived from the same organism as the other sequence.

An isolated or substantially pure nucleic acid molecule or polynucleotide is a polynucleotide which is substantially separated from other polynucleotide sequences which naturally accompany a native transcription regulatory sequence. The term embraces a polynucleotide sequence which has been removed from its naturally occurring environment, and includes recombinant or cloned DNA isolates, chemically synthesized analogues and analogues biologically synthesized by heterologous systems.

A polynucleotide is said to encode a polypeptide if, when manipulated by methods known to those skilled in the art, it can be transcribed and/or translated to produce the polypeptide or a fragment thereof.

A nucleotide sequence is operably linked when it is placed into a functional relationship with another nucleotide sequence. For instance, a promoter is operably linked to a coding sequence if the promoter effects its transcription or expression. Generally, operably linked means that the sequences being linked are contiguous and, where necessary to join two protein coding regions, contiguous and in reading frame. However, it is well known that certain genetic elements, such as enhancers, may be operably linked even at a distance, i.e., even if not contiguous.

The term recombinant polynucleotide refers to a polynucleotide which is made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

Large amounts of the polynucleotides may be produced by replication in a suitable host cell. Natural or synthetic DNA fragments coding for a protein of interest are incorporated into recombinant polynucleotide constructs, typically DNA constructs, capable of introduction into and replication in a prokaryotic or eukaryotic cell, especially a mammalian cell in culture, e.g., Vero cells, CHO cells, among others, or Aspergillus pullulans or Aspergillus nidulans, Trichoderma reesei, Saccharomyces cerevisiae or Pichia pastoris, where protein expression is desired. Usually the construct is suitable for replication in a unicellular host, such as a bacterium, but a multicellular eukaryotic host may also be appropriate, with or without integration within the genome of the host cell. Commonly used prokaryotic hosts include strains of Escherichia coli, although other prokaryotes, such as Bacillus subtilis or a pseudomonad, may also be used. Such factors as ease of manipulation, ability to appropriately glycosylate expressed proteins, degree and control of protein expression, ease of purification of expressed proteins away from cellular contaminants or other factors influence the choice of the host cell.

The polynucleotides may also be produced by chemical synthesis, e.g., by the phosphoramidite method described by Beaucage and Caruthers (1981) Tetra. Letts. 22: 1859-1862 or the triester method according to Matteuci et al. (1981) J. Am. Chem. Soc. 103: 3185, and may be performed on commercially available, automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

DNA constructs prepared for introduction into a prokaryotic or eukaryotic host will typically comprise a replication system (i.e. vector) recognized by the host, including the intended DNA fragment encoding the desired polypeptide, and will preferably also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides may also be included where appropriate from secreted polypeptides of the same or related species, which allow the protein to cross and/or lodge in cell membranes or be secreted from the cell.

An appropriate promoter and other necessary vector sequences will be selected so as to be functional in the host. Examples of workable combinations of cell lines and expression vectors are described in Sambrook et al. (1989) vide infra; Ausubel et al. (Eds.) (1995) Current Protocols in Molecular Biology, Greene Publishing and Wiley Interscience, New York; and Metzger et al. (1988) Nature, 334: 31-36. Many useful vectors for expression in bacteria, yeast, fungal, mammalian, insect, plant or other cells are well known in the art and may be obtained such vendors as Stratagene, New England Biolabs, Promega Biotech, and others. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, N.Y. (1983). While such expression vectors may replicate autonomously, they may less preferably replicate by being inserted into the genome of the host cell.

Expression and cloning vectors advantageously contain a selectable marker, that is, a gene encoding a protein necessary for the survival or growth of a host cell transformed with the vector. Although such a marker gene may be carried on another polynucleotide sequence co-introduced into the host cell, it is most often contained on the cloning vector. Only those host cells into which the marker gene has been introduced will survive and/or grow under selective conditions. Typical selection genes encode proteins that confer resistance to antibiotics or other toxic substances, e.g., ampicillin, neomycin, methotrexate, etc.; complement auxotrophic deficiencies; or supply critical nutrients not available from complex media. The choice of the proper selectable marker will depend on the host cell; appropriate markers for different hosts are known in the art.

Recombinant host cells, in the present context, are those which have been genetically modified to contain an isolated DNA molecule of the instant invention. The DNA can be introduced by any means known to the art which is appropriate for the particular type of cell, including without limitation, transformation, lipofection or electroporation.

It is recognized by those skilled in the art that the DNA sequences may vary due to the degeneracy of the genetic code and codon usage. All DNA sequences which code for the polypeptide or protein of interest are included in this invention.

Additionally, it will be recognized by those skilled in the art that variations may occur in the coding sequence specifically exemplified herein which will not significantly change activity of the amino acid sequence of the encoded polypeptide. All such equivalent DNA sequences are included within the scope of this invention and the definition of the regulated promoter region. The skilled artisan will understand that the sequence of the exemplified sequence can be used to identify and prepare additional, nonexemplified nucleotide sequences which are functionally equivalent to the sequences given.

Thus, mutational, insertional, and deletional variants of the disclosed nucleotide sequence can be readily prepared by methods which are well known to those skilled in the art. These variants can be used in the same manner as the exemplified primer sequences so long as the variants have substantial sequence homology with the original sequence. As used herein, substantial sequence identity refers to identity which is sufficient to enable the variant polynucleotide to function in the same capacity as the specifically exemplified Ecto-NOX specific single chain antibody. Preferably, this identity is greater than 85%, even more preferably this identity is greater than 90%, and most preferably, this identity is greater than 95%. It is well within the skill of a person trained in this art to make mutational, insertional, and deletional mutations which are equivalent in function or are designed to improve the function of the sequence or otherwise provide a methodological advantage. Substitutions, insertions or deletions of from 1 to 5 amino acids which do not affect the binding specificity are within the scope of the present invention. Methods for confirming antigen (Ecto-NOX tNOX) binding specificity are well known in the art.

Polymerase Chain Reaction (PCR) is a repetitive, enzymatic, primed amplification of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). By using a thermostable DNA polymerase such as the Taq polymerase, which is isolated from the thermophilic bacterium Thermus aquaticus, the amplification process can be completely automated. Other enzymes which can be used are known to those skilled in the art. It is understood that PCR can also be used to join fragments of DNA and/or to introduce defined mutations into a sequence of interest. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu. Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein. Antiobody vaccines are described in Dillman R. O. (2001) Cancer Invest. 19(8):833-841. Durrant L. G. et al. (2001) Int. J. Cancer 1; 92(3):414-420 and Bhattacharya-Chatterjee M. (2001) Curr. Opin. Mol. Ther. Feb. 3(1):63-69 describe anti-idiotype antibodies. Many of the procedures useful for practicing the present invention whether or not described herein in detail, are well known to those skilled in the arts of molecular biology, biochemistry, immunology, and medicine.

All references cited in the present application are incorporated by reference herein to the extent that there is no inconsistency with the present disclosure. Such references reflect the skill in the arts relevant to the present invention.

The following examples are provided for illustrative purposes, and are not intended to limit the scope of the invention as claimed herein. Any variations in the exemplified single chain antibodies, coding and amino acid sequences, epitopes, purification methods, diagnostic methods, preventative methods, treatment methods, and other methods which occur to the skilled artisan are intended to fall within the scope of the present invention.

### EXAMPLES

### Example 1

### Recombinant tNOX-Specific Single Chain Antibody

Monoclonal antibody generated against tNOX NADH oxidase tumor cell specific was produced in sp-2 myeloma cells, however, the monoclonal antibody slowed the growth of sp-2 myeloma cells that were used for fusion with spleen cells after 72 h. This phenomenon made it difficult to produce antibody in quantity. tNOX-specific monoclonal antibody-producing cells are described in U.S. Pat. No. 7,053,188, and they are on deposit with the American Type Culture Collection as PTA-4206. To overcome this problem, the coding sequences of the antigen-binding variable region of the heavy chain and the light chain (Fv region) of the antibody cDNA were cloned and linked into one chimeric gene, upstream of the S-tag coding sequence. The Fv portion of an antibody, consisting of variable heavy (V_{H}) and variable light (V_{L}) domains, can maintain the binding specificity and affinity of the original antibody (Glockshuber et al. 1990. Biochemistry 29:1262-1367).

### Example 2

### Cloning cDNAs Encoding the Variable Regions of Immunoglobulin Heavy Chain and Light Chain

For a recombinant antibody, cDNAs encoding the variable regions of immunoglobulin heavy chain (V_{H}) and light chain (V_{I}), are cloned by using degenerate primers. Mammalian immunoglobulins of light and heavy chain contain conserved regions adjacent to the hypervariable complementary defining regions (CDRs). Degenerate oligoprimer sets allow these regions to be amplified using PCR (Jones et al. 1991. Bio/Technology 9:88-89; Daugherty et al. 1991. Nucleic Acids Research 19:2471-2476). Recombinant DNA techniques have facilitated the stabilization of variable fragments by covalently linking the two fragments by a polypeptide linker (Huston et al. 1988. Proc. Natl. Acad. Sci. USA 85:5879-5883). Either V_{L} or V_{H} can provide the NH₂-terminal domain of the single chain variable fragment (ScFv). The linker should be designed to resist proteolysis and to minimize protein aggregation. Linker length and sequences contribute and control flexibility and interaction with ScFv and antigen. The most widely used linkers have sequences consisting of glycine (Gly) and serine (Ser) residues for flexibility, with charged residues as glutamic acid (Glu) and lysine (Lys) for solubility (Bird et al. 1988. Science 242:423-426; Huston et al. 1988. supra).

### Example 3

### Isolation of RNA

Total RNA was isolated from the hybridoma cells (ATCC Accession No. PTA-4206) producing tNOX-specific monoclonal antibodies by the following procedure modified from Chomczynski et al. (1987) Anal. Biochem. 162:156-159 and Gough (1988) Anal. Biochem 176:93-95. Cells were harvested from medium and pelleted by centrifugation at 450×g for 10 min. Pellets were gently resuspended with 10 volumes of ice cold PBS and centrifuged again. The supernatant was discarded and cells were resuspended with an equal volume of PBS. Denaturing solution (0.36 ml of 2-mercaptoethanol/50 ml of guanidinium stock solution-4M guanidinium thiocyanate, 25 mM sodium citrate, pH 7.0, 0.5% sarkosyl) 10 ml per 1 g of cell pellet was added prior to use and mixed gently. Sodium acetate (pH 4.0,1 ml of 2M), 10 ml of phenol saturated water and 2 ml of chloroform:isoamyl alcohol (24:1) mixtures were sequentially added after each addition. The solution was mixed thoroughly by inversion. The solution was vigorously shaken for 10 sec, chilled on ice for 15 min and then centrifuged 12,000×g for 30 min. The supernatant was transferred and an equal volume of 2-propanol was added and placed at −20° C. overnight to precipitate the RNA. The RNA was pelleted for 15 min at 12,000×g, and the pellet was resuspended with 2-3ml of denaturing solution and 2 volumes of ethanol. The solution was placed at −20° C. for 2 h, and then centrifuged at 12,000×g for 15 min. The RNA pellet was washed with 70% ethanol and then 100% ethanol. The pellet was resuspended with RNase-free water (DEPC-treated water) after centrifugation at 12,000×g for 5 min. The amount of isolated RNA was measured spectrophotometrically and calculated from the absorbance at 280 nm and 260 nm.

The poly(A)mRNA isolation kit was purchased from Stratagene. Total RNA was applied to an oligo(dT) cellulose column after heating the total RNA at 65° C. for 5 min. Before applying, the RNA samples were mixed with 500 μl of 10×sample buffer (10 mM Tris-HCl, pH 7.5,1 mM EDTA, 5 M NaCl). The RNA samples were pushed through the column at a rate of 1 drop every 2 sec. The eluates were pooled and reapplied to the column and purified again. Preheated elution buffer (65° C.) was applied, and mRNA was eluted and collected in 1.5 ml of centrifuge tubes on ice. The amount of mRNA was determined at OD₂₆₀ (1 OD unit=40 μg of RNA). The amounts of total RNA and mRNA obtained from 4×10⁸ cells were 1328 μg and 28 μg, respectively.

mRNA (1-2 μg) dissolved in DEPC-treated water was used for cDNA synthesis. mRNA isolated on three different dates was pooled for first-strand cDNA synthesis. The cDNA synthesis kit was purchased from Pharmacia Biotech. mRNA (1.5 μg/5 μl of DEPC-treated water) was heated at 65° C. for 10 min. and cooled immediately on ice. The primed first strand mix containing MuLV reverse transcriptase (11 μl) and appropriate buffers for the reaction were mixed with mRNA sample. DTT solution (1 μl of 0.1 M) and RNase-free water (16 μl) also were added to the solution. The mixture was incubated for 1 h at 37° C.

### Example 4

### ScFv Primer Design

Degenerate primers for light chain and heavy chain (Novagen, Madison, Wis.) were used for PCR. PCR synthesis was carried out in 100 μl reaction volumes in 0.5 ml microcentrifuge tubes by using Robocycler (Stratagene, La Jolla, Calif.). All PCR syntheses included 2 μl of sense and anti-sense primers (20 pmoles/μl ), 1 μl of first-strand cDNA as a template, 2 μl of 10 mM of dNTPs, 1 μl of Vent polymerase (2 units/μl), 10 μl of 10×PCR buffer (100 mM Tris-HCl, pH 8.8 at 25° C., 500 mM KCl, 15 mM MgCl₂, 1% Triton X-100), 82 μl of H₂O. Triton X-100 is t-octylphenoxypolyethoxyethanol. All PCR profiles consisted of 1 min of denaturation at 94° C., 1 min of annealing at 55° C., and 1 min of extension at 72° C. This sequence was repeated 30 times with a 6-min extension at 72° C. in the final cycle. PCR products were purified with QIAEX II gel extraction kit from Qiagen, Valencia, Calif. PCR amplification products for heavy and light chain coding sequences were analyzed by agarose gel electrophoresis and were about 340 base pair (bp) long and 325 bp long, respectively.

Total RNA or DNA was analyzed by agarose gel electrophoresis (1% agarose gels). Agarose (0.5 g in 50 ml of TAE buffer, 40 mM Tris-acetate, 1 mM EDTA) was heated for 2 min in a microwave to melt and evenly disperse the agarose. The solution was cooled at room temperature, and ethidium bromide (0.5 μg/ml) was added and poured into the apparatus. Each sample was mixed with 6×gel loading buffer (0.25% bromophenol blue, 0.25% xylene cyanol FF, 40% (w/v) sucrose in water). TAE buffer was used as the running buffer. Voltage (10 v.cm) was applied for 60-90 min.

### Example 5

### PCR Amplification

According to the proper size for heavy and light chain cDNAs, the bands were excised from the gels under UV illumination, and excised gels were placed in 1 ml syringes fitted with 18-gauge needles. Gels were crushed to a 1.5 ml Eppendorf tube. The barrel of each syringe was washed with 200 μl of buffer-saturated phenol (pH 7.9±0.2). The mixture was thoroughly centrifuged and frozen at −70° C. for 10 min. The mixture was centrifuged for 5 min, and the top aqueous phase was transferred to a new tube. The aqueous phase was extracted again with phenol/chloroform (1:1). After centrifuging for 5 min, the top aqueous phase was transferred to a clean tube, and chloroform extraction was performed. Sodium acetate (10 volumes of 3 M) and 2.5 volumes of ice-cold ethanol were added to the top aqueous phase to precipitate DNA at −20° C. overnight.

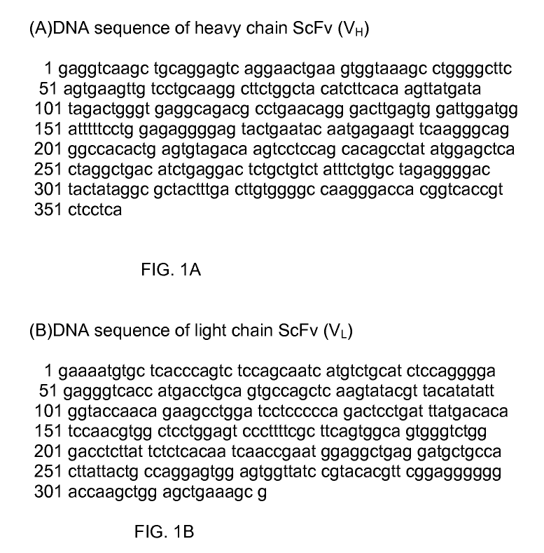

Purified heavy and light chain cDNAs were ligated into plasmid pSTBlue-1 vector and transfected into NovaBlue competent cells (Stratagene). Colonies containing heavy and light chain DNAs were screened by blue and white colony selection and confirmed by PCR analysis. Heavy and light chain DNAs were isolated and sequenced using standard techniques. FIGS. 1A and 1B show the DNA sequences of heavy and light chain DNAs of ScFv.

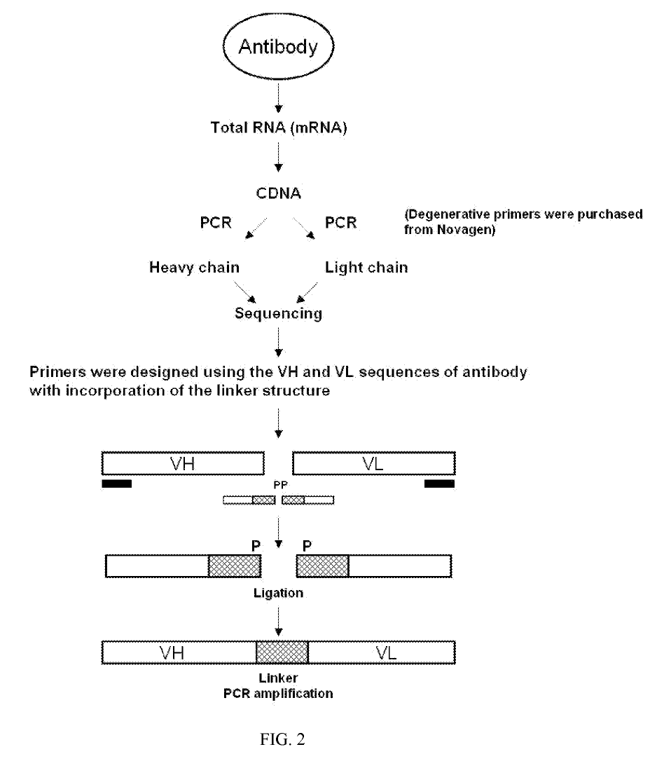

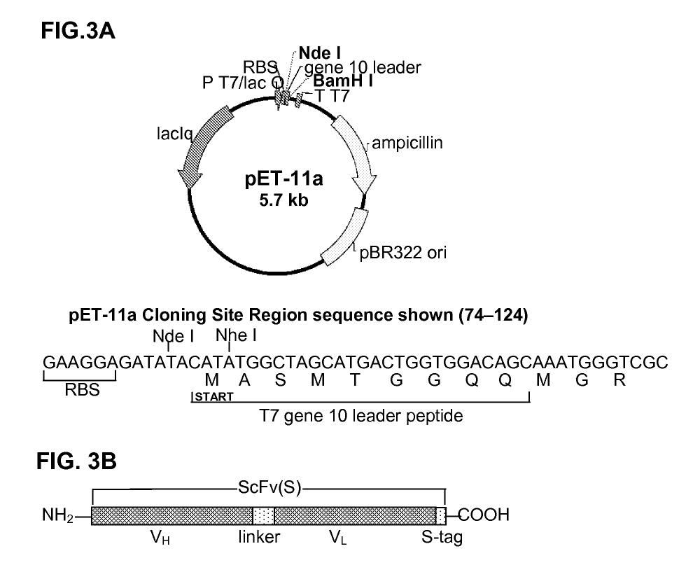

PCR amplification and the assembly of single ScFv gene was according to Davis et al. (1991) Bio/Technology 9:165-169 (FIG. 2). Plasmid pSTBlue-1 carrying V_{H} and V_{L} genes were combined with all four oligonucleotide primers in a single PCR synthesis (FIG. 3). Following first PCR synthesis, one tenth of the first PCR product was removed and added to a second PCR reaction mixture containing only the primer a (V_{H} sense primer) and primer d (V_{L} Antisense primer). The product of the second PCR synthesis yielded single ScFv gene. The single ScFv gene was ligated to plasmid pT-Adv (Clontech, Palo Alto, Calif.). pT-Adv carrying ScFv gene was used for DNA sequencing.

### Example 6

### Linker Design

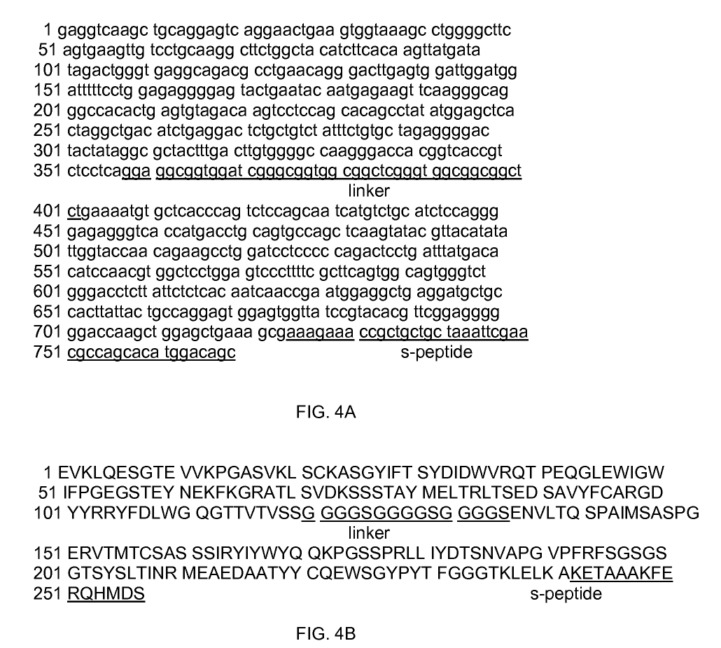

The complete ScFv gene was assembled from the V_{H}, V_{L} and linker genes to yield a single ScFv gene by PCR (FIG. 4). The DNA sequence encoding the linker was 45 nucleotides long (GGAGGCGGTGGATCGGGCGGTGGCGGCTCGGGTGGCGGCGGCTCT; SEQ ID NO:6), which translates to a peptide of 15 amino acids (GlyGlyGlyGlySerGlyGlyGlyGlySerGlyGlyGlyGlySer; SEQ ID NO:5). Primers for PCR amplification are shown in Table 3. S-peptide was linked to the C-terminus of ScFv[ScFv(S)]. S-peptide binds to S-protein conjugated to alkaline phosphatase for Western blot analysis. The DNA sequence of the S-peptide is AAAGAAACCGCTGCTGCTAAATTCGAACGCCAGCACATGGACAGC (SEQ ID NO:7) which translates to S-peptide (LysGluThrAlaAlaAlaLysPheGluArgGlnHisMetAspSer; SEQ ID NO:8).

### Example 7

### Expression of Recombinant ScFv

Recombinant ScFv(S) was expressed in E. coli. First, oligo nucleotides encoding S-peptide were linked to the 3′ end of the open reading frame (ORF) of ScFv DNA by PCR amplification. Incorporation of S-peptide enables to detect expressed ScFv protein by S-protein conjugated to alkaline phosphatase. The tNOX-specific ScFv(S) coding sequence was then subcloned to plasmid pET-11a, a plasmid designed for protein expression in E. coli (Stratagene, Calif.). For PCR amplification, two primers were designed to amplify ORF of ScFv(S) containing endonuclease restriction sites (NdeI and NheI) and S-peptide residues.

Plasmid pET-11a and ORF of ScFv(S) were digested with restriction enzymes NdeI and NhI and ligated to produce plasmid pET11-ScFv(S). E. coli BL21 (DE3) was transformed with pET11-ScFv(S) and grown at 37° C. for 12 h in LB medium containing ampicillin (100 μg/ml). ScFv was expressed by addition of 0.5 mM IPTG and incubation for 4 h. Cells were harvested and lysed using a French Pressure Cell (French Pressure Cell Press, SLM Instruments, Inc.) (three passages at 20,000 psi). Cell extracts were centrifuged at 10,000×g for 20 min. Pellets containing denatured inclusion bodies of ScFv were collected. Renaturation of the inclusion bodies containing the ScFv was according to Goldberg et al. 1995. Folding & Design 1:21-27.

TABLE 1
Primers for PCR amplification of ScFv(s) gene

| | |
|---|---|
| 1. | Primers for cloning of variable regions of |
| | heavy chain and light chain of antibody |
| (A) | Primers for heavy chain (V_{H}) |
| | Forward primer: |
| | 5′-GGCCCAGCCGGCCGAGGTCAAGCTGCAGGAGTCAGGA-3′ |
| | (SEQ ID NO: 9) |
| | Reverse primer: |
| | 5′-CTCGGAACCTGAGGAGACGGTGACCGTGGTCCC-3′ |
| | (SEQ ID NO: 10) |
| (B) | Primers for light chain (V_{L}) |
| | Forward primer: |
| | 5′-TCCAAAGTCGACGAAAATGTGCTCACCCAGTCTCCA-3′ |
| | (SEQ ID NO: 11) |
| | Reverse primer: |
| | 5′-AGCGGCCGCTTTCAGCTCCAGCTTGGTCCCCCC-3′ |
| | (SEQ ID NO: 12) |
| 2. | Primers for subcloning of ScFv(s) gene into |
| | pET-11a expression vector |
| (A) | Primers for heavy chain (V_{H}) and linker |
| | amplification |
| | Forward primer: |
| | 5′-GTCAAGCTGCAGGAGTCAGGA-3′ |
| | (SEQ ID NO: 13) |
| | Reverse primer: |
| | 5′-AGAGCCGCCGCCACCCGAGCCGCCACCGCCCGATCCACCGCCTC |
| | CTGAGGAGACGGTGACCGTGGT-3′ |
| | (SEQ ID NO: 14) |
| (B) | Primers for light chain (V_{L}), linker and S-tag |
| | amplification |
| | Forward primer: |
| | 5′-GGAGGCGGTGGATCGGGCGGTGGCGGCTCGGGTGGCGGCGGCTC |
| | TGAAAATGTGCTCACCCAGTCT-3′ |
| | (SEQ ID NO: 15) |
| | Reverse primer: |
| | 5′-AGTCAGGCTAGCTTAGCTGTCCATGTGCTGGCGTTCGAATTTAG |
| | CAGCAGCGGTTTCTTTCGCTTTCAGCTCCAGCTT-3′ |
| | (SEQ ID NO: 16) |

### BIBLIOGRAPHY

1. Morré, D. J. (1998) in Plasma Membrane Redox Systems and Their Role in Biological Stress and Disease (Asard, H., Bérczi, A. and Caubergs, R. J., Eds) pp. 121-156, Kluwer Academic Publishers, Dordrecht, Netherlands.
2. Morré, D. J. and Morré, D. M. (2003) Free Radical Res. 37: 795-808
3. Morré, D. J., Chueh, P.-J. and Morré, D. M. (1995) Proc. Natl. Acad. Sci. USA 92:1831-1835.
4. Bruno, M., Brightman, A. O., Lawrence, J., Werderitsh, D., Morré, D. M. and Morré, D. J. (1992) Biochem. J. 284: 625-628.
5. Cho, N., Chueh, P.-J., Kim, C., Caldwell, S., Morré, D. M. and Morré, D. J. (2002) Cancer Immunol. Immunother. 51: 121-129.
6. Morré, D. J., Caldwell, S., Mayorga, A., Wu, L-Y. and Morré, D. M. (1997) Arch. Biochem. Biophys. 342: 224-230.
7. Morré, D. J. and Reust, T. (1997) J. Bioenerg. Biomemb. 29, 281-289.
8. Morré, D. J. (1995) Biochim. Biophys. Acta 1240: 201-208.
9. del Castillo-Olivares, A., Chueh, P.-J., Wang, S., Sweeting, M., Yantiri, F., Sedlak, D., Morré, D. J. and Morré, D. M. (1998) Arch. Biochem. Biophys. 358: 125-140.
10. Morré, D. J., Sedlak, D., Tang, X., Chueh, P. J., Geng, T. and Morré, D. M. (2001) Arch. Biochem. Biophys. 392: 251-256.
11. Chueh, P. J., Kim, C., Cho, N., Morré, D. M. and Morré, D. J. (2002) Biochemistry 41: 3732-3741.
12. Bird, C. (1999) Direct submission of human DNA sequence from clone 875H3 (part of APK1 antigen) to GenBank database at NCBI. (Accession no. AL049733).

What is claimed is:

1. A nucleic acid molecule comprising a coding sequence for a single chain antibody which specifically reacts with ECTO-NOX proteins, said coding sequence comprising the variable light chain coding sequence and a variable heavy chain sequence of a tNOX-specific monoclonal antibody expressed by a hybridoma cell line on deposit with the American Type Culture Collection as Accession No. PTA-4206, and a coding sequence for linker of the amino acid sequence GGGGSGGGGSGGGGS (SEQ ID NO:5) covalently linked in frame between said variable heavy chain coding sequence and said light chain coding sequence.

2. The nucleic acid molecule of claim 1, further comprising a sequence encoding an protein tag.

3. The nucleic acid molecule of claim 2, wherein said protein tag is an S tag.

4. The nucleic acid molecule of claim 2, wherein said protein tag is a polyhistidine protein tag, a calmodulin tag, maltose binding protein tag, a Nus protein tag, Myc protein tag, flagellar antigen tag, streptavidin tag, or a glutathione S-transferase tag.

5. The nucleic acid molecule of claim 3 encoding a single chain antibody which specifically reacts with ECTO-NOX proteins, wherein said single chain antibody comprises the amino acid sequence set forth in SEQ ID NO:4.

6. The nucleic acid molecule of claim 5, wherein the coding sequence for said single chain antibody is as set forth in SEQ ID NO:3.

7. The nucleic acid molecule of claim 1, wherein the coding sequence is operably linked to transcription control sequences.

8. The nucleic acid molecule of claim 7, wherein said molecule further comprises vector sequences.

9. A host cell comprising the nucleic acid molecule of claim 7.

10. A method for recombinantly expressing a single chain antibody which specifically reacts with ECTO-NOX proteins, comprising the step of culturing the host cell of claim 9.

11. The method of claim 10, further comprising the step of recovering the expressed single chain antibody.

12. A method for joining an antibody light chain variable region and an antibody heavy chain variable region, said method comprising the step of inserting a coding sequence for a peptide linker consisting of the amino acid sequence GGGGSGGGGSGGGGS (SEQ ID NO:5) in frame between a coding sequence for an antibody light chain variable region and a coding sequence for an antibody heavy chain variable region.


SEQUENCE LISTING (18 sequences)

SEQ ID NO: 1 - DNA, 357 bp, Artificial
  Synthetic construct  coding sequence of heavy chain ScFV (VH)
gaggtcaagctgcaggagtcaggaactgaagtggtaaagcctggggcttcagtgaagttgtcctgcaaggcttctggctacatcttcacaagttatgatatagactgggtgaggcagacgcctgaacagggacttgagtggattggatggatttttcctggagaggggagtactgaatacaatgagaagttcaagggcagggccacactgagtgtagacaagtcctccagcacagcctatatggagctcactaggctgacatctgaggactctgctgtctatttctgtgctagaggggactactataggcgctactttgacttgtggggccaagggaccacggtcaccgtctcctca

SEQ ID NO: 2 - DNA, 321 bp, Artificial
  Synthetic construct  coding sequence of light chain ScFv (VL)
gaaaatgtgctcacccagtctccagcaatcatgtctgcatctccaggggagagggtcaccatgacctgcagtgccagctcaagtatacgttacatatattggtaccaacagaagcctggatcctcccccagactcctgatttatgacacatccaacgtggctcctggagtcccttttcgcttcagtggcagtgggtctgggacctcttattctctcacaatcaaccgaatggaggctgaggatgctgccacttattactgccaggagtggagtggttatccgtacacgttcggaggggggaccaagctggagctgaaagcg

SEQ ID NO: 3 - DNA, 768 bp, Artificial
  Synthetic construct  coding sequence of TNOX-specific ScFv (single chain antibody)
gaggtcaagctgcaggagtcaggaactgaagtggtaaagcctggggcttcagtgaagttgtcctgcaaggcttctggctacatcttcacaagttatgatatagactgggtgaggcagacgcctgaacagggacttgagtggattggatggatttttcctggagaggggagtactgaatacaatgagaagttcaagggcagggccacactgagtgtagacaagtcctccagcacagcctatatggagctcactaggctgacatctgaggactctgctgtctatttctgtgctagaggggactactataggcgctactttgacttgtggggccaagggaccacggtcaccgtctcctcaggaggcggtggatcgggcggtggcggctcgggtggcggcggctctgaaaatgtgctcacccagtctccagcaatcatgtctgcatctccaggggagagggtcaccatgacctgcagtgccagctcaagtatacgttacatatattggtaccaacagaagcctggatcctcccccagactcctgatttatgacacatccaacgtggctcctggagtcccttttcgcttcagtggcagtgggtctgggacctcttattctctcacaatcaaccgaatggaggctgaggatgctgccacttattactgccaggagtggagtggttatccgtacacgttcggaggggggaccaagctggagctgaaagcgaaagaaaccgctgctgctaaattcgaacgccagcacatggacagc

SEQ ID NO: 4 - protein, 256 aa, Artificial
  Synthetic construct  amino acid sequence of ScFv (single chain antibody) specific for tNOX, ECTO-NOX
EVKLQESGTEVVKPGASVKLSCKASGYIFTSYDIDWVRQTPEQGLEWIGWIFPGEGSTEYNEKFKGRATLSVDKSSSTAYMELTRLTSEDSAVYFCARGDYYRRYFDLWGQGTTVTVSSGGGGSGGGGSGGGGSENVLTQSPAIMSASPGERVTMTCSASSSIRYIYWYQQKPGSSPRLLIYDTSNVAPGVPFRFSGSGSGTSYSLTINRMEAEDAATYYCQEWSGYPYTFGGGTKLELKAKETAAAKFERQHMDS

SEQ ID NO: 5 - protein, 15 aa, Artificial
  Synthetic construct  linker sequence for joining heavy and light chains in single chain antibody specific for tNOX (ECTO-NOX)
GGGGSGGGGSGGGGS

SEQ ID NO: 6 - DNA, 45 bp, Artificial
  Synthetic construct  oligonucleotide sequence encoding linker
ggaggcggtggatcgggcggtggcggctcgggtggcggcggctct

SEQ ID NO: 7 - DNA, 45 bp, Artificial
  Synthetic construct  oligonucleotide encoding S peptide
aaagaaaccgctgctgctaaattcgaacgccagcacatggacagc

SEQ ID NO: 8 - protein, 15 aa, Artificial
  Synthetic construct  amino acid sequence of S peptide
KETAAAKFERQHMDS

SEQ ID NO: 9 - DNA, 37 bp, Artificial
  Synthetic construct  oligonucleotide useful as a primer
ggcccagccggccgaggtcaagctgcaggagtcagga

SEQ ID NO: 10 - DNA, 33 bp, Artificial
  Synthetic construct  oligonucleotide useful as a primer
ctcggaacctgaggagacggtgaccgtggtccc

SEQ ID NO: 11 - DNA, 36 bp, Artificial
  Synthetic construct  oligonucleotide useful as a primer
tccaaagtcgacgaaaatgtgctcacccagtctcca

SEQ ID NO: 12 - DNA, 33 bp, Artificial
  Synthetic construct  oligonucleotide useful as a primer
agcggccgctttcagctccagcttggtcccccc

SEQ ID NO: 13 - DNA, 21 bp, Artificial
  Synthetic construct  oligonucleotide useful as a  primer
gtcaagctgcaggagtcagga

SEQ ID NO: 14 - DNA, 66 bp, Artificial
  Synthetic construct  oligonucleotide useful as a primer
agagccgccgccacccgagccgccaccgcccgatccaccgcctcctgaggagacggtgaccgtggt

SEQ ID NO: 15 - DNA, 66 bp, Artificial
  Synthetic construct  oligonucleotide useful as a primer
ggaggcggtggatcgggcggtggcggctcgggtggcggcggctctgaaaatgtgctcacccagtct

SEQ ID NO: 16 - DNA, 78 bp, Artificial
  Synthetic construct  oligonucleotide useful as a primer
agtcaggctagcttagctgtccatgtgctggcgttcgaatttagcagcagcggtttctttcgctttcagctccagctt

SEQ ID NO: 17 - DNA, 51 bp, Artificial
  Synthetic construct  sequence encoding T7 gene 10 leader peptide in expression vector.
gaaggagatatacatatggctagcatgactggtggacagcaaatgggtcgc

SEQ ID NO: 18 - protein, 12 aa, Artificial
  Synthetic construct  T7 gene 10 leader peptide with three additional amino acids.
MASMTGGQQMGR